(19) United States
(12) Patent Application Publication
(10) Pub. No.: US 2022/0183537 A1
(43) Pub. Date: Jun. 16, 2022

(54) A COMMUNICATION MODULE FOR AN IN-VIVO DEVICE

(71) Applicant: Given Imaging LTD, Yoqneam (IL)

(72) Inventors: Iddo Diukman, Zihron Yaakov (IL); Baruch Gruman, Lapid (IL)

(21) Appl. No.: 17/442,758

(22) PCT Filed: Mar. 25, 2020
(86) PCT No.: PCT/IL2020/050358

§ 371 Date: Sep. 24, 2021

Related U.S. Application Data
(60) Provisional application No. 62/824,680, filed on Mar. 27, 2019.

(51) Int. Cl. A61B 1/00; H04B 5/00; A61B 1/04

(52) U.S. Cl. CPC A61B 1/00016; A61B 1/00009; A61B 1/041; H04B 5/0031

(57) ABSTRACT

An ex-vivo communication module configured for communicating with a swallowable in-vivo device. The communication module comprises a receiving unit operating at a first frequency range and configured for receiving signals from the in-vivo device, and a transmitting unit operating at a second frequency range, different from the first frequency range and configured for transmitting signals to the in-vivo device. The transmitting unit is also configured for serving as a secondary receiving unit, receiving signals from the in-vivo device.

### TECHNOLOGICAL FIELD

The present invention is in the field of communication and transmission, in particular, referring to systems configured for communicating with a swallowable in-vivo device.

### BACKGROUND OF THE INVENTION

It is well known in the art to use swallowable in-vivo devices to monitor and detect pathologies in the GI tract. In its most common form, the in-vivo device comprises an in-vivo communication module configured for transmitting and receiving signals, for example, transmitting images of the GI tract captured by the in-vivo device and receiving orders relating to its operation.

The in-vivo device usually operates in conjunction with an ex-vivo device comprising an ex-vivo communication module, the in-vivo and ex-vivo communication modules forming together a communication system.

The ex-vivo device can be a portable device fitted to the patient to be in the closest possible proximity to the GI tract and the general area in which the in-vivo device is expected to be.

Acknowledgement of the above references herein is not to be inferred as meaning that these are in any way relevant to the patentability of the presently disclosed subject matter.

### SUMMARY

In accordance with one embodiment of the subject matter of the present application, there is provided an ex-vivo communication module configured for communicating with a swallowable in-vivo device, said communication module comprising a receiving unit configured for operating at a first frequency range for receiving signals from said in-vivo device, and a transmitting unit configured for operating at a second frequency range, different from said first frequency range (e.g. not including overlapping frequencies), for transmitting signals to said in-vivo device, wherein said transmitting unit also constitutes a secondary receiving unit, configured for receiving signals from the in-vivo device.

Hereinafter, the terms ‘uplink unit’ and ‘downlink unit’ may be used interchangeably with the terms ‘receiving unit’ and ‘transmitting unit’. Specifically, the term ‘downlink’ refers to transmitting a signal to the in-vivo device while the term ‘uplink’ refers to receiving a signal from the in-vivo device.

The communication module is configured for being located external to the body, for example, placed on the body of the patient, for allowing proper communication between the module and the in-vivo device.

The receiving unit and the transmitting unit may be constituted by antennas, each antenna being designed for its own operation frequency range. In accordance with one example, the first frequency range may be at least one order of magnitude greater/smaller than the second frequency range. The transmitting unit may be configured for operating at 5-30 MHz, more specifically at 10-20 MHz, and even more specifically at 12-15 MHz, while the receiving unit may be configured for operating at 350-550 Mhz, more specifically at 400-500 MHz, and even more specifically at 420-450 MHz.

It should be appreciated that since the ex-vivo module and the in-vivo device need to communicate with each other, it would be desired to increase the transmission quality by using a low frequency range, both for the uplink and for the downlink, since low frequency range transmissions have a lower attenuation within the human body. However, since the in-vivo device is required to transmit a large amount of data (e.g. images obtained from the GI), using a low frequency as in the downlink antenna, may not provide sufficient bandwidth for such an amount of data. Therefore, while the downlink antenna may still be chosen to be at the above mentioned low frequency range of several MHz, the uplink antenna is chosen to be at a high frequency range of several hundred MHz.

In accordance with the above, it should be appreciated that the design of the downlink antenna is optimized for its specific frequency range, and would therefore not be expected to perform properly for receiving transmissions at a considerably different frequency range, e.g. as previously mentioned, one or more orders of magnitude higher/lower. Nonetheless, it is suggested, under the concept of the subject matter of the present application, to use the downlink antenna as a secondary uplink (receiving) antenna, contradictory to its originally intended design.

Owing to the unique arrangement of the above system, requiring close proximity between the ex-vivo device and the in-vivo device, using the downlink antenna as a secondary receiver was found surprisingly useful in providing at least some of the necessary uplink communication with the in-vivo device at the high frequency range.

In other words, while the frequency range of the downlink antenna is around 13 MHz, and naturally, if used as a receiving unit, would not be useful as an antenna at the high frequency range (around 400 MHz), it still functions as a receiving antenna at a close range, albeit with poorer results. Thus, the above communication module provides a configuration in which two antennas, designed for operation at two different frequency ranges (high frequency and low frequency) and for two different purposes, both cover the receiving end of an uplink communication with the in-vivo device (which transmits at a single predetermined high frequency range), thereby compensating for each other, posing a significant improvement in uplink communication.

In operation, the in-vivo device traveling along the GI tract of the patient constantly changes its distance from, and orientation with respect to, the ex-vivo communication module. While the transmissions of the in-vivo device are to be received by the uplink unit, the position and orientation of the in-vivo device may, at times, be such that the uplink unit does not properly receive the transmitted signal. It is at this point that the downlink unit comes into effect as a secondary uplink receiving unit, operating as a backup/complementary receiving unit. Thus, even if the uplink signal received by the downlink unit is of poor quality, it may still be better than receiving no signal at all by the uplink unit alone.

It is appreciated that at some instances, neither the downlink unit nor the uplink unit may receive any signal from the in-vivo device. However, during testing of the above uplink/downlink configuration, it was demonstrated that using the downlink unit as an uplink backup provides receiving a greater percentage of the in-vivo transmissions compared to the standard configuration, in which only the uplink unit is used for uplink reception.

The communication module may comprise a processor configured for providing input to the transmitting unit (which will then be transmitted to the in-vivo device), and for receiving data from the receiving unit. The communication module may further comprise a modem unit interposed between the processor and the receiving/transmitting unit, configured for providing communication therebetween.

The modem unit may be configured for applying a diversity scheme, whereby it detects which of the units provides a better uplink transmission, and, upon such detection, chooses the better transmission of the two to be provided to the processor. In this manner, the quality of the data received from the in-vivo device may be optimized.

In accordance with one example, the uplink unit may have a dual connection with the modem, one for uplink and one for downlink, and the modem may be configured for continuously alternating between a downlink mode, providing data to the downlink unit and an uplink mode, receiving data from said uplink unit. Alternatively, in accordance with another example, both the uplink connection and the downlink connection of the uplink unit may be connected to the modem via a multiplexer, allowing continuous uplink/downlink communication between the modem and the uplink/downlink units.

The communication module may be incorporated within an ex-vivo device configured for being fitted to the patient. In accordance with one example, the ex-vivo device may be a patch configured for being applied to the patient's skin. In accordance with another example, the ex-vivo device may be a portable device configured for being carried by the patient, similar to a monitor. In accordance with still another example, the ex-vivo device may be a belt configured for being fitted to the patient and extend around his/her body, rather than having a pin-point location.

The uplink and downlink antennas may be flat, allowing at least part of the communication module to have a flat-sheet design, making it particularly suitable for incorporation into a patch or belt as suggested above. Specifically, a flat-sheet design of the communication module may allow it to bend and twist, thereby allowing it to assume the natural shape of the patient's body.

In accordance with one specific example, the communication module may have the shape of a rectangular flat-sheet design, having a length dimension L and a width dimension W. Under this design, the receiving antenna may be formed on the flat-sheet while the transmitting antenna may extend circumferentially along the edge of the flat-sheet. Specifically, the receiving antenna may be a monopole antenna.

In accordance with a specific design, the transmitting antenna may extend around the monopole uplink antenna, and be a coil-antenna, making several loops around the receiving antenna.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic view of a patient's body fitted with a patch comprising the communication module of the present application;

FIG. 1B is a schematic view of the GI tract of the patient from FIG. 1A, containing an in-vivo device configured for communicating with the communication module of FIG. 1A;

FIG. 2 is a schematic view of the communication module shown in FIG. 1A;

FIG. 3 is a schematic view of the patch shown in FIG. 1A, comprising the communication module of the present application;

FIG. 4A is a schematic isometric view of a another example of a patch, comprising a communication module in accordance with the present application;

FIG. 4B is a schematic exploded view of the layers comprising the patch shown in FIG. 4A; and

FIG. 4C is a schematic front view of the communication module incorporated in the patch shown in FIGS. 4A and 4B.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS

In the following detailed description, numerous specific details are set forth in order to provide a thorough understanding of the invention. However, it will be understood by those skilled in the art that the present invention can be practiced without these specific details. In other instances, well-known methods, procedures, and components, modules, units and/or circuits have not been described in detail so as not to obscure the invention.

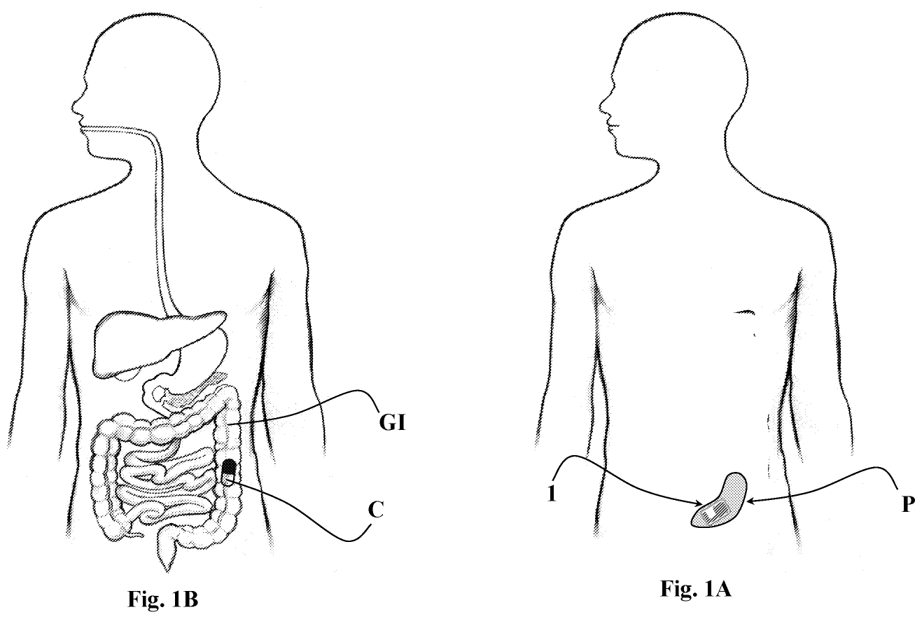

Attention is first drawn to FIGS. 1A and 1B, in which a patient is shown having an in-vivo device in the form of a swallowable capsule C contained within their GI tract, and being fitted, externally, with a patch P comprising a communication module of the present application, generally designated 1.

The communication module 1 is configured for communicating with the swallowable capsule C during its travel along the GI tract, both receiving data from the capsule C (referred herein as ‘uplink’) and transmitting data to the capsule C (referred herein as ‘downlink’). Specifically, the uplink data may contain images captured by the capsule C, parameters recorded thereby etc., while downlink data may include instructions sent to the capsule, for example, in order to change an operation mode of the capsule C, change its frame rate etc.

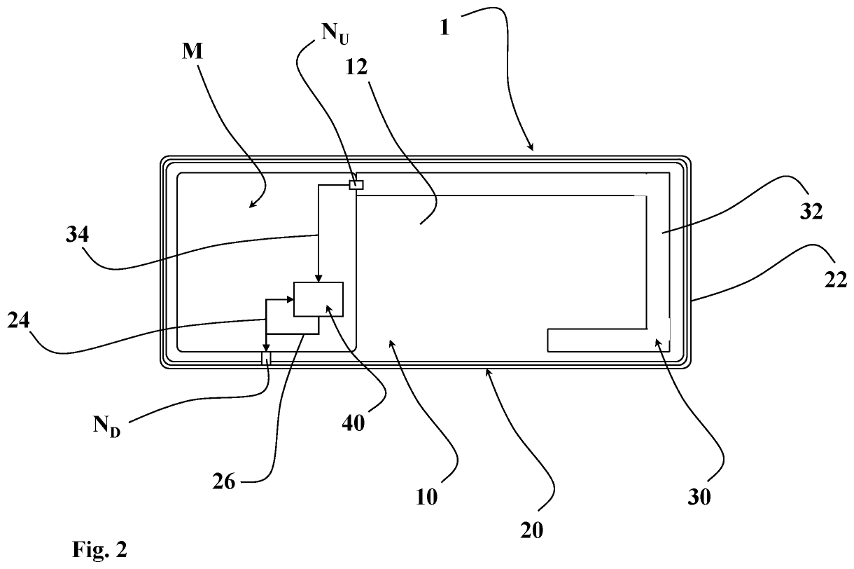

Turning now to FIG. 2, the communication module 1 is shown comprising a base 10 made of a flexible sheet of material 12 which is fitted with a board M, and having imprinted thereon a downlink antenna 20 extending circumferentially around the base 10 and an uplink antenna 30. The board M further comprises a modem unit 40 associated with both the uplink antenna 30 and the downlink antenna 20 via respective connections N_{U} and N_{D}.

The downlink antenna 20 is in the form of an antenna coil 22 and is configured for transmitting data to the capsule C at around 13.5 MHz. The uplink antenna 30 is in the form of a monopole antenna 32 and is configured for receiving transmissions from the capsule C at around 435 MHz. In addition, the downlink antenna 20 is also configured for receiving transmission from the capsule C, thereby operating as a secondary uplink antenna.

It should be noted that the capsule C is designed to transmit data at a high frequency range (hundreds of MHz), owing to bandwidth considerations, yielded by the requirement to transmit a large amount of data (e.g. in-vivo images). Therefore, the uplink antenna 30 is chosen to operate at a corresponding high frequency range, for optimizing reception from the capsule C. However, the downlink antenna 20 is not similarly limited in its transmission frequency, and can therefore be designed to operate at a low frequency range (tens of MHz), which reduces attenuation of the signal while passing through the tissue of the human body.

During travel of the capsule C within the GI tract, it may, at times, reach a location, assume a position or orientation which lead to the uplink antenna 30 being unable to properly receive the signal transmitted by the capsule C. In order to compensate for this, the downlink antenna 20 is used, despite being optimized for operating at a frequency range considerably different than that of the capsule's transmitter, and may even be able to better pick up the signal from the capsule C than the uplink antenna 30.

While the modem unit 40 is connected to the uplink antenna 30 only as a receiver via connection 34, it is connected to the downlink antenna 20 both as a transmitter via link 26 and as a receiver via link 24. Each of the uplink/downlink antennas 30, 20, may pick up a strong signal, a weak signal or no signal at all. The modem unit 40 is configured for operating under a diversity scheme, detecting which of the connections 24, 34 provides the stronger signal and prefer it to the weaker signal, leading to at least the following cases (the terms ‘weak’ and ‘strong’ used herein are used relatively to one another):

TABLE 1

| | Uplink antenna | Downlink antenna | Modem unit selects |
|---|---|---|---|
| 1 | Strong signal | No signal | Signal from uplink antenna |
| 2 | Strong signal | Weak signal | Signal from uplink antenna |
| 3 | Weak signal | No signal | Signal from uplink antenna |
| 4 | Weak signal | Strong signal | Signal from downlink antenna |
| 5 | No signal | Strong signal | Signal from downlink antenna |
| 6 | No signal | Weak signal | Signal from downlink antenna |
| 7 | No signal | No signal | None |

It was clearly demonstrated, during testing of the above uplink/downlink configuration, that using the downlink unit as an uplink backup provides, statistically, receiving a greater percentage of the transmissions from the in-vivo capsule C compared to a configuration in which the downlink antenna is only used for downlink.

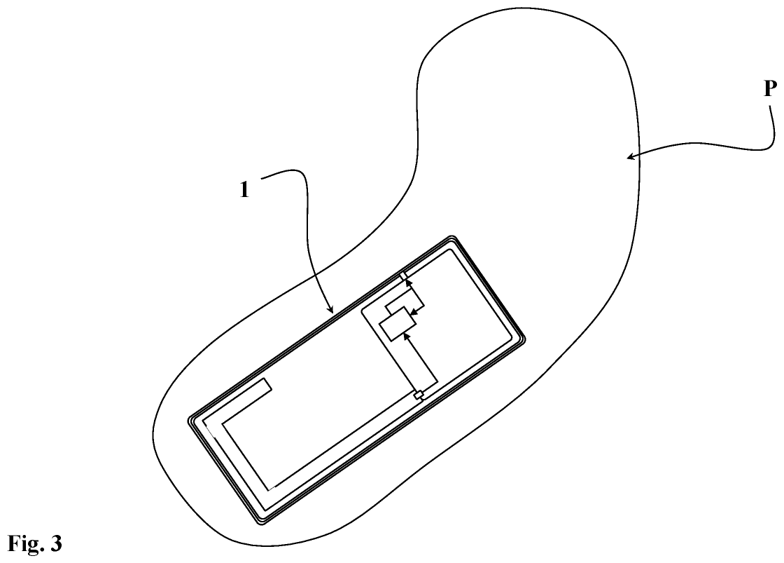

Turning now to FIG. 3, the communication module 1 may be incorporated within a patch P, which is, in turn, configured for being fitted to a patient's body by, for example, adhesion. It should be noted that the flexibility of the sheet 12 (and of the antennas 20, 30 printed thereon) may provide a significant advantage in terms of user comfort, since the patch is to be adhered to the body and thus provides a less limited movement on the side of the patient.

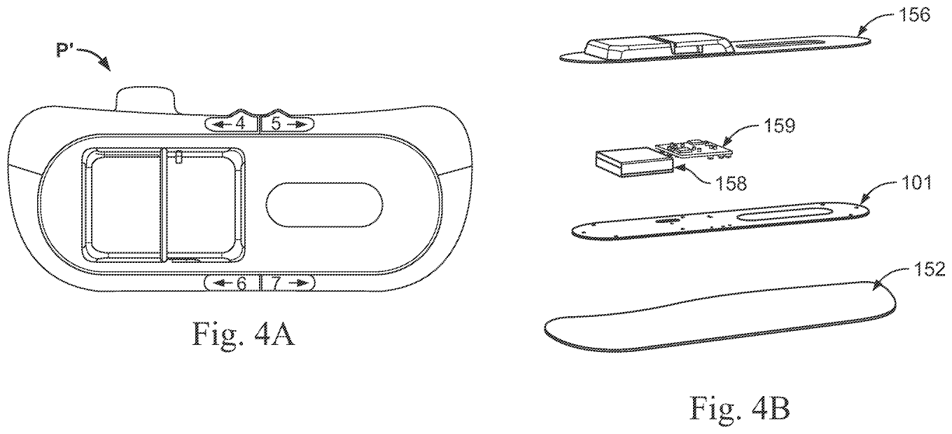

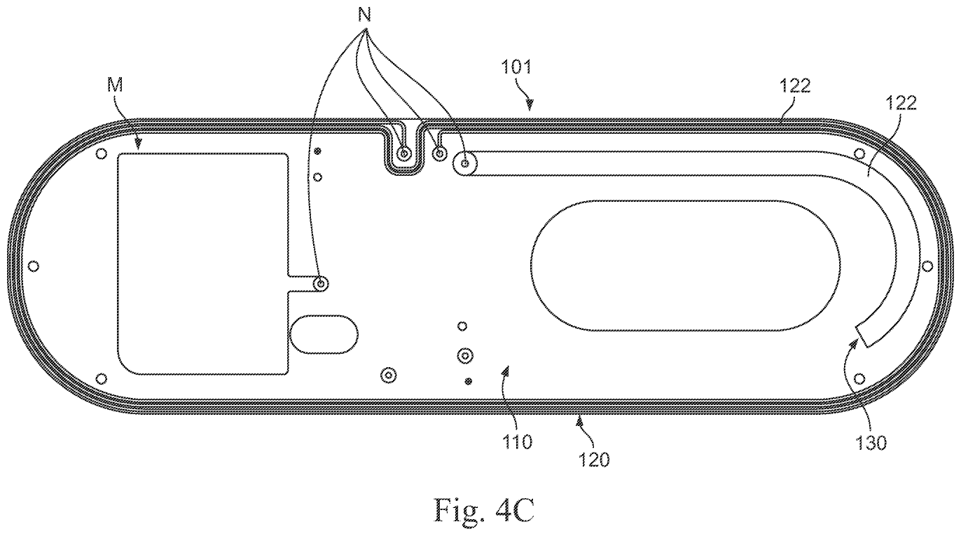

Turning now to FIGS. 4A to 4C, another example of a patch according to some embodiments is shown, generally designated P′, and including a plurality of functional layers including (but not limited to):
an adhesive layer 152 configured for being in direct contact with the patient's body and for fixating the position of the patch with respect to the patient's body;
a communication layer 101 constituting the communication module; and
an external cover layer 156;

The patch 10 further comprises a power unit 158 and a processing unit 159 nested within the external cover layer 156.

With particular reference being drawn to FIG. 4C, the scheme of the communication module 101 is shown comprising an oval base 110 made of a flexible sheet of material 112 which is fitted with a board M, and having imprinted thereon a downlink antenna 120 extending circumferentially around the oval base 110 and an uplink antenna 130. The board M and antennas 120, 130 are configured for being connected with one another via connection ends N.

The communication module 101 is essentially similar to the previously communication module 1, with the difference lying mostly in the oval design of the printed antenna (compared to the rectangular design of communication module 1), and in the design of the patch P′.

Those skilled in the art to which this invention pertains will readily appreciate that numerous changes, variations, and modifications can be made without departing from the scope of the invention, mutatis mutandis.

It will thus be seen that the objects set forth elsewhere herein, among those made apparent from the preceding description, are efficiently attained and, because certain changes may be made in carrying out the method described elsewhere herein and in the construction(s) set forth without departing from the spirit and scope of the invention, it is intended that all matter contained in the above description and shown in the accompanying drawings shall be interpreted as illustrative and not in a limiting sense.

In the foregoing detailed description, numerous specific details are set forth in order to provide an understanding of the invention. However, it will be understood by those skilled in the art that the invention can be practiced without these specific details. In other instances, well-known methods, procedures, and components, modules, units and/or circuits have not been described in detail so as not to obscure the invention. Some features or elements described with respect to one embodiment can be combined with features or elements described with respect to other embodiments.

Although embodiments of the invention are not limited in this regard, the terms “plurality” and “a plurality” as used herein can include, for example, “multiple” or “two or more”. The terms “plurality” or “a plurality” can be used throughout the specification to describe two or more components, devices, elements, units, parameters, or the like. The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

It is also to be understood that the following claims are intended to cover all of the generic and specific features of the invention herein described and all statements of the scope of the invention which, as a matter of language, might be said to fall therebetween.

1. An ex-vivo communication module configured for communicating with a swallowable in-vivo device, the communication module comprising:
a receiving unit configured for operating at a first frequency range for receiving signals from the in-vivo device; and
a transmitting unit configured for operating at a second frequency range, different from the first frequency range, for transmitting signals to the in-vivo device, wherein the transmitting unit also constitutes a secondary receiving unit configured for receiving signals from the in-vivo device.

2. The communication module according to claim 1, wherein the communication module is configured for being placed on the body of the patient, for allowing communication between the communication module and the in-vivo device.

3. The communication module according to claim 1, wherein the receiving unit comprises an antenna and the transmitting unit comprises an antenna, each antenna configured to operate at its own frequency range.

4. The communication module according to claim 3, wherein the first frequency range is at least one order of magnitude greater or smaller than the second frequency range.

5. The communication module according to claim 1, wherein the transmitting unit is configured for operating at 5-30 MHz.

6. The communication module according to claim 4, wherein the receiving unit is configured for data transfer operating at 350-550 Mhz.

7. (canceled)

8. The communication module according to claim 1, wherein the communication module includes a processor configured for at least one of:
(a) providing input to the transmitting unit; or
(b) receiving data from the receiving unit.

9. The communication module according to claim 8, wherein the communication module includes a modem unit configured for providing communication between the processor and at least one of the receiving unit or the transmitting unit.

10. The communication module according to claim 9, wherein the communication module includes an uplink unit configured to provide communication between the processor and one of the receiving unit or the transmitting unit based on a comparison between an uplink transmission provided by the receiving unit and an uplink transmission provided by the transmitting unit.

11. The communication module according to claim 10, wherein the uplink unit includes a downlink unit configured for downlink communication with the modem unit, and an uplink unit configured for uplink communication with the modem unit.

12. The communication module according to claim 11, wherein the modem unit is configured for continuously alternating between a downlink mode, providing data to the downlink unit and an uplink mode, receiving data from the uplink unit.

13. The communication module according to claim 11, wherein the uplink unit and the downlink unit are connected to the modem unit via a multiplexer, allowing continuous communication between the modem unit and the uplink and downlink units.

14. (canceled)

15. The communication module according to claim 1, wherein the communication module is a patch configured for being applied to the patient's skin.

16. The communication module according to claim 1, wherein the communication module is a portable device configured for being carried by the patient.

17. The communication module according to claim 1, wherein the communication module is a belt configured for being fitted to the patient.

18. The communication module according to claim 1, wherein the uplink and downlink units are flat, and are incorporated in a flexible sheet of the communication module.

19. The communication module according to claim 18, wherein the flexible sheet allows the communication module to assume the natural shape of the patient's body.

20-24. (canceled)

25. An ex-vivo communication module configured for communicating with a swallowable capsule, the communication module comprising:
a receiving unit configured to operate within a first frequency range for receiving image data from the swallowable capsule while the swallowable capsule travels within a patient's GI tract;
a transmitting unit configured to operate within a second frequency range, different from the first frequency range, to at least one of:
transmit signals to the swallowable capsule while the swallowable capsule travels within the patient's GI tract; or
operate as a secondary receiving unit for receiving the image data from the swallowable capsule while the swallowable capsule travels within the patient's GI tract; and
a modem unit configured to:
detect the strength of a first communication signal between the receiving unit and the swallowable capsule;
detect the strength of a second communication signal between the transmitting unit and the swallowable capsule; and
cause one of the receiving unit or the transmitting unit to receive the image data from the swallowable capsule based on a comparison between the strength of the first communication signal and the strength of the second communication signal.

26. An ex-vivo communication module configured for communicating with a swallowable capsule, the communication module comprising:
an uplink antenna configured to operate within a first frequency range for receiving an uplink signal from the swallowable capsule while the swallowable capsule travels within a patient's GI tract; and
a downlink antenna configured to operate within a second frequency range, different from the first frequency range, to at least one of:
transmit a downlink signal to the swallowable capsule while the swallowable capsule travels within the patient's GI tract; or
receive the uplink signal from the swallowable capsule while the swallowable capsule travels within the patient's GI tract based on a comparison between the strength of a communication signal between the uplink antenna and the swallowable capsule and the strength of a communication signal between the downlink antenna and the swallowable capsule.

27. The communication module according to claim 26, further comprising a modem unit configured to cause one of the uplink antenna or the downlink antenna to receive the uplink signal from the swallowable capsule based on the comparison.
